(19) United States
(12) Patent Application Publication
(10) Pub. No.: US 2023/0379596 A1
(43) Pub. Date: Nov. 23, 2023

(54) LIGHT FIELD ENDOSCOPE WITH LOOK AROUND CAPABILITY

(71) Applicant: Asensus Surgical US, Inc., Durham, NC (US)

(72) Inventor: Randall L. Luck, Cary, NC (US)

(21) Appl. No.: 18/092,295

(22) Filed: Dec. 31, 2022

Related U.S. Application Data
(60) Provisional application No. 63/295,703, filed on Dec. 31, 2021.

(51) Int. Cl. H04N 23/957; H04N 13/383; H04N 23/50; H04N 13/302; H04N 13/243; A61B 1/00

(52) U.S. Cl. CPC H04N 23/957; H04N 13/383; H04N 23/555; H04N 13/302; H04N 13/243; A61B 1/00193; A61B 1/0008; H04N 13/398

(57) ABSTRACT

A multi-camera, multi-view light field endoscope simultaneously captures images of a body cavity using a plurality of camera sensors. The system makes use of input from an eye and head tracker to generate control signals selecting the specific pair of camera sensors that correspond to the user's eye and head position. By selecting only signals from the selected pair, transmission bandwidth, data rate, and system complexity are reduced. Use of the eye and head tracker give the user a look around, autostereoscopic capability with a simple, commercially available stereo display.

This application claims the benefit of U.S. Provisional Application No. 63/295,703, filed Dec. 31, 2021.

### BACKGROUND

This applications describes a video endoscope system with 3D and “look around capabilities”. The components include a multi-view light field endoscope, a standard stereo LCD monitor, eyewear, an eye and head position tracker, and control software.

We see the world in three dimensions. We have two eyes that see the world at slightly different horizontal positions. Our brains interpret this slight horizontal visual disparity (parallax) as three-dimensional depth. People have been interested in recreating this capability with art, photography, and video since at least the 16th century. All such attempts involve presenting two views, one to each eye. First attempts used two hand drawn pictures. Starting in the mid-19th century, Wheatstone, Brewster, and Holmes developed techniques and viewers using two photographs. A dual camera was used which recorded the two views. These systems used special viewers called stereoscopes which focused and presented each image, one to each eye. The viewer's brain then merges these two pictures giving depth perception. By the early to mid-20th century, stereo (3-D) movies were developed. The first of these used anaglyphic techniques. A special movie camera filmed two images side by side in synchronism. After film processing and editing, then they were projected onto a movie screen. In the projector, one of the views was passed through a cyan filter and the other view was passed through a red filter. The audience wore special glasses, the lens for one eye was cyan and the lens for the other eye was red. The viewer's brain then merged these cyan and red images, giving depth perception. Color rendition was fair at best.

By the latter part of the 20th century, video systems were developed. The first such systems used a standard CRT display and active eye wear. A special electronic switch arrangement alternated the right and left image frames at a rate faster than perceptible, typically the 60 Hz video frame rate. The switch also provided a signal to control special eyewear worn by each user. The eye wear had an optical shutter that alternately passed or blocked the right or left image in synchronism with the video frames. The viewer's brain then merges these two alternating images giving depth perception.

By the early 2000's projection displays and flat panel liquid crystal displays with passive eye wear were developed. The right and left images are displayed with alternating optical polarization. Users wear glasses that have one lens polarized in one direction and the other lens polarized at 90 degrees from the first. As a result, one eye only sees one view and the other eye only sees the other view. The user's brain merges these two views, giving depth perception. All commercial movies that are now being presented in “3-D” use this type of system. Many surgical endoscope systems also use this system. See FIG. 1A. Stereo displays only ever present a right-left pair of images. With any of those described above, when the user moves their head from side to side, the depth image gives the impression of “following” their motion. They can never see behind objects as would be possible in a natural scene.

Other means of presenting depth involving several different but conceptually similar methods were developed by Lippmann, F. E. Ives, H. E. Ives, and others from the start of the 20th century into the 1930's. These systems used parallax barriers, multiple slit cameras, or lenticular sheets to capture multiple views of a scene at the same time. Recording and display systems were film based and primarily optical, but they provided multiple views out into the viewing area at the same time. More than one person could experience the display, and each had their own unique view. H. E. Ives developed several film projectors that could display as many as 39 images at the same time onto a lenticular screen. See, for example, U.S. Pat. No. 1,883,291. Due to the primitive nature of electronics at the time, these were very limited systems. Static, printed lenticular 3D pictures can still be seen today on things like novelty birthday cards, post cards, and advertising pieces.

Autostereoscopy is a method of displaying stereoscopic images. It does not require special headgear or glasses, etc., in order for the user to be able to perceive the images in 3D. See FIG. 1E. As optics and electronics became more advanced starting in the early 1960's, such autostereoscopic systems became more sophisticated. Collender, Burckhardt, DeMontebello, and Okoshi described several early systems. Okoshi 1976, Three-Dimensional Imaging Techniques, Academic Press, U.S. Pat. Nos. 3,178,720, 4,089,597. Such display systems offered look around capabilities but are severely limited by bandwidth and image storage space. They are also usually very complex. In the example of Ives system, since there are 39 cameras and 39 projectors, the system requires 39 times as much film. If such a system were to be built today using modern electronics, 39 video cameras, 39 video projectors, 39 times the bandwidth, 39 times the number of cables, and 39 times the storage space would be needed compared with a simple monocular system. Take for example a monocular system with 16 bit color, 1920×1080 HD video at 60 frames per second. This requires a data rate on the order of 2 gigabits per second and one minute of uncompressed video requires a storage space of 120 gigabits. With 39 views, the data rate is about 78 gigibits per second and 1 minute of such video requires a storage space of about 6.24 terabits. Because of these severe conditions, in general, from the 1980's until recently, most research in autostereo has been limited to display techniques with computer generated images.

See also Levoy and Hanrahan 1996, Light Field Rendering, Proceedings of SIGGRAPH '96. Jones et al 2014 describe a system that uses 72 projectors. Jones, Nagano, Liu, Busch, Yu, Bolas, and Debevec 2014, Interpolating vertical parallax for an autostereoscopic three-dimensional projector array, Journal of Electronic Imaging 23.

Modern day two-channel prior art endoscope systems are depicted in FIGS. 1A-1B. In the FIG. 1A system, there are two image sensors and each image is ultimately sent to the right and left channel inputs of the stereo monitor. Passive polarized eye wear is typically used but active or anaglyphic eye wear could be used. In this set up, the user perceives depth, but they have no look around capability. No matter where they place their head relative to the display, they will always see the same depth image.

The FIG. 1B system also makes use of two image sensors in the endoscope but this time each is sent to a separate small video monitor. Folding and focusing optics send the right image into the right eye and the left image into the left eye. No eye wear is used, but instead the user's head must be placed into the viewer. Notice that this kind of system is essentially a modern, real time video version of the old-fashioned stereoscope viewers that date back to the 19th century. There is no look around capability and in this case the user's head is in a fixed position and head motion is very limited.

FIG. 1C illustrates a stereo LCD display that tracks a single user's head position. The monitor is configured to present the right image to the right eye and the left to the left eye without using eye wear, providing glasses-free depth perception but no look around capability. Thus, the user sees the same depth image regardless of where their head is positioned. See U.S. Pat. No. 8,456,516. Some patents (U.S. Pat. Nos. 5,311,220, 5,349,379, 9,992,485 are a few for examples) describe an autostereoscopic screen that similarly presents direction selective light to an observer's right and left eyes under control of a head position tracker. These displays are autostereoscopic and do not require the use of special eye wear. These displays lack solutions to issues surrounding bandwidth, data rates, and complexity between the multi-camera and the display.

As has been demonstrated, the bandwidth and storage requirements of autosteroscopic systems are severe.

A primary objective of this disclosure is therefore to preserve the look around capability of a minimal autostereoscopic system while having the bandwidth, simplicity, and lower cost of a simple two channel stereoscopic system.

### BRIEF DESCRIPTION OF THE DRAWINGS

FIGS. 1A-1B are schematic diagrams illustrating prior art stereo endoscope display configurations.

FIG. 1C is a schematic diagram illustrating a prior art “glasses-less” stereo image display configuration.

FIG. 1D is a schematic diagram illustrating a prior art autostereoscopic image display configuration.

FIG. 2 is a perspective view of an embodiment of a light field endoscope camera, shown in the deployed system.

FIGS. 3A-3C are a sequence of side elevation views showing the light field endoscope camera of FIG. 2 in a stowed configuration, an insertion configuration, and a deployed configuration, respectively.

FIG. 4 is a plan view of a light field endoscope display arrangement that may be used with the light field endoscope camera of FIG. 2.

FIG. 5 is a schematic diagram illustrating capture and display of images using the embodiment of FIGS. 2-3C.

FIG. 6A is similar to FIG. 5 and depicts display of images from image pair A-B based on the user eye and head position. FIG. 6B shows the resulting left eye image and right eye image.

FIGS. 7A and 7B are similar to FIGS. 6A and 6B, but depict display of images from image pair B-C based on the user eye and head position.

FIGS. 8A and 8B are similar to FIGS. 6A and 6B, but depict display of images from image pair C-D based on the user eye and head position.

FIGS. 9A and 9B are similar to FIGS. 6A and 6B, but depict display of images from image pair D-E based on the user eye and head position.

FIGS. 10A and 10B are similar to FIGS. 6A and 6B, but depict display of images from image pair E-F based on the user eye and head position.

FIGS. 11A-11F display a sequence of views of a pair of objects as seen by a user when altering the users head position while observing display during use of the disclosed system.

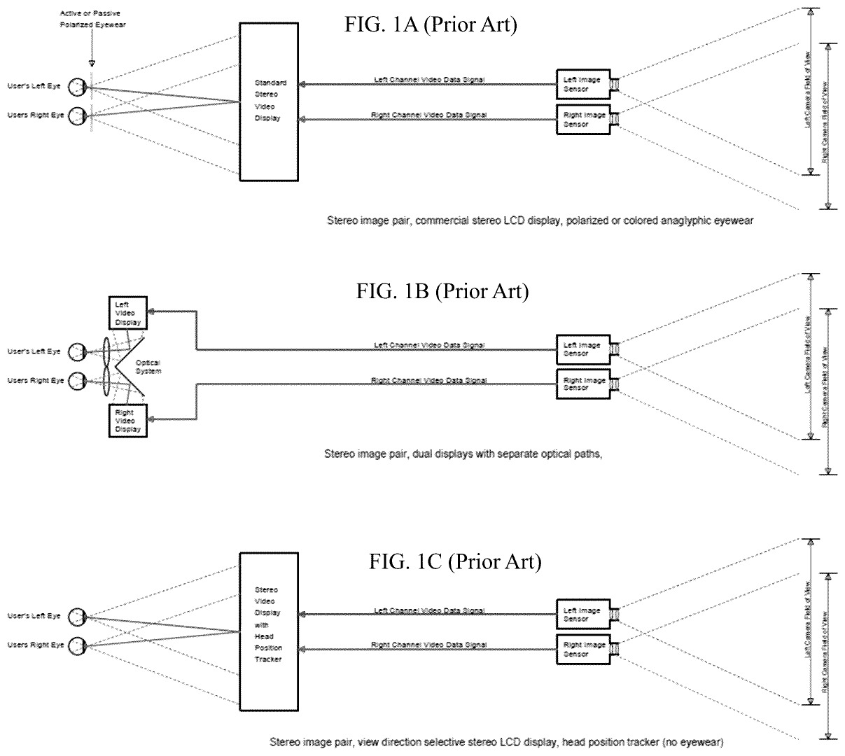

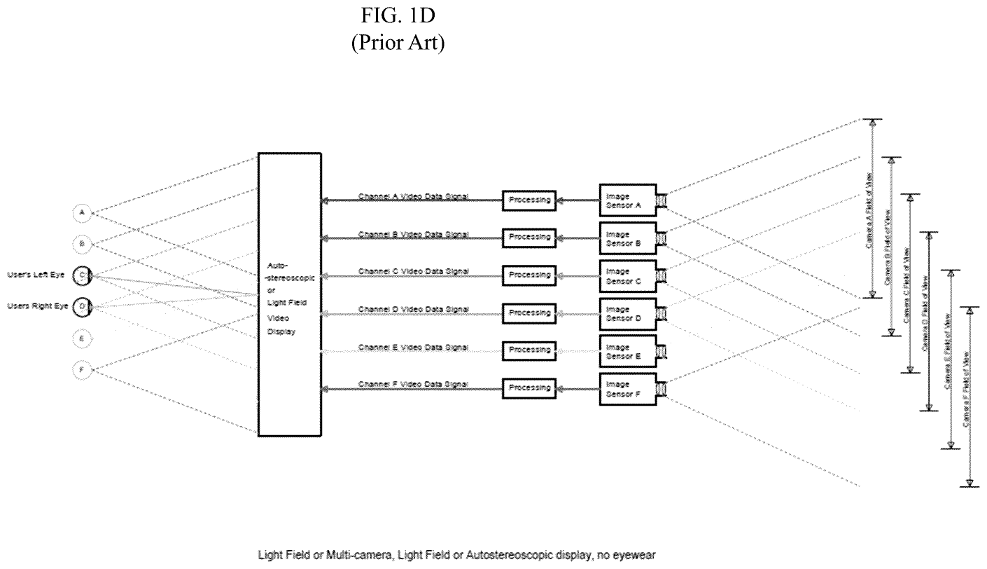

### DETAILED DESCRIPTION

The present application describes an autostereoscopic or light field endoscopic camera system that preserves the look around capability of prior autostereoscopic systems while having the bandwidth, simplicity, and lower cost of a simple two channel stereoscopic system. The described concept allows for a light field camera to be made in endoscopic form, meeting the reduced cabling requirements that are beneficial and practical to endoscope systems, where all signals need to pass through a small incision, natural orifice, or trocar port in the patient, which may be restricted to a diameter of 10 mm or less.

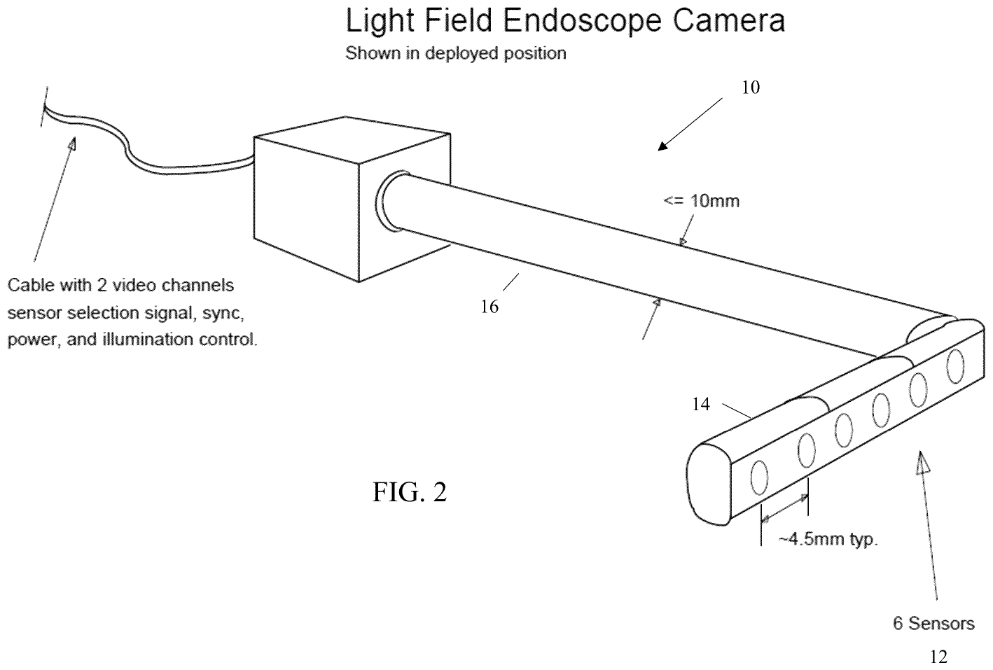

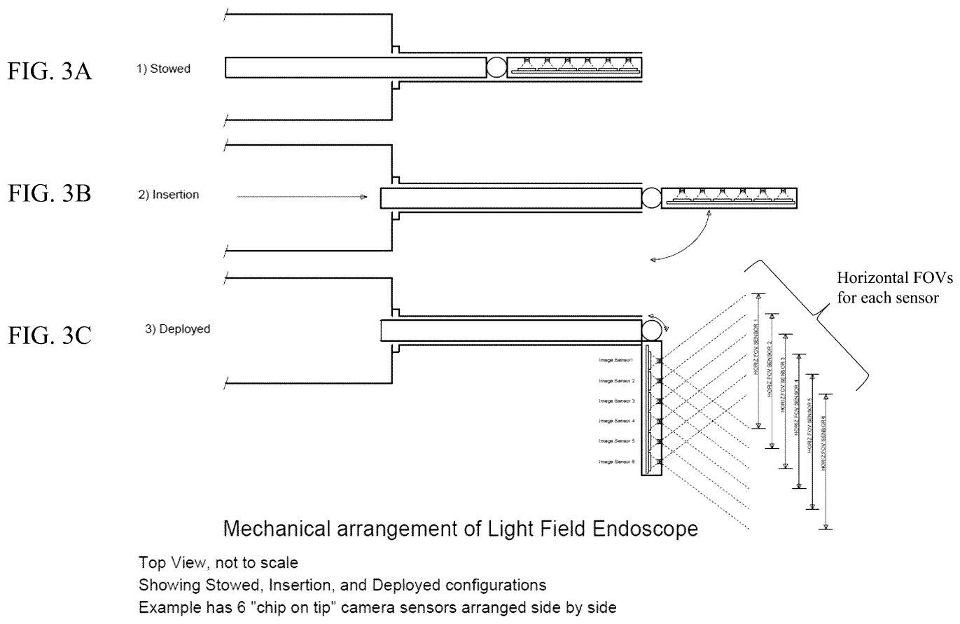

Referring to FIGS. 2-4, an embodiment of a light field endoscope includes the following components.
1) A multi-camera light field endoscope.
2) A video switching system.
3) Connecting cables.
4) A standard stereo LCD or projection display.
5) Polarized eye wear for use with the stereo display.
6) An eye and head tracker device.
7) Control software, firmware, or hardware.

In the embodiment shown in the drawing, the example multi-camera endoscope 10 is equipped with six image camera sensors 12, although other larger or smaller numbers of sensors may be suitable. For example, three or more, four or more, five or more, or six or more sensors are used.

The camera sensors may be the type of image sensors commonly used for endoscope or laparoscopic images, such as CCD sensors, CMOS sensors, or other images sensors known now or developed in the future.

The camera sensors are positioned side by side so as to product six views with horizontal disparity. A desirable spacing for the sensors is 4 to 5 mm center-to-center as depicted in FIG. 2, however alternate spacing may be used.

To facilitate introduction into a body cavity, the camera sensors are positioned on a camera head 14 that articulates relative to the fixed elongate shaft 16. Again, for an endoscope, this means that the sensor mounting must enter the patient's port sideways. After introduction into the patient, the head 14 articulates 90 degrees relative to the shaft 16 or associated trocar. In the embodiment shown in the drawings, the head 14 and shaft 16 are rigid members having a mechanical hinge, vertebrae section, or bendable structure between them. In other embodiments, one or both of the head 14 and shaft 16 (or the portion of the shaft just proximal to the head 14) may be made of flexible material or a vertebrae configuration.

While it may be preferable to position the camera sensors on the camera head, alternatives in which the camera sensors are positioned more proximally in the camera where they receive light captured via lenses at the camera head (e.g. in an arrangement similar to that shown for the distal sensors) from related optical components are considered within the scope of this disclosure.

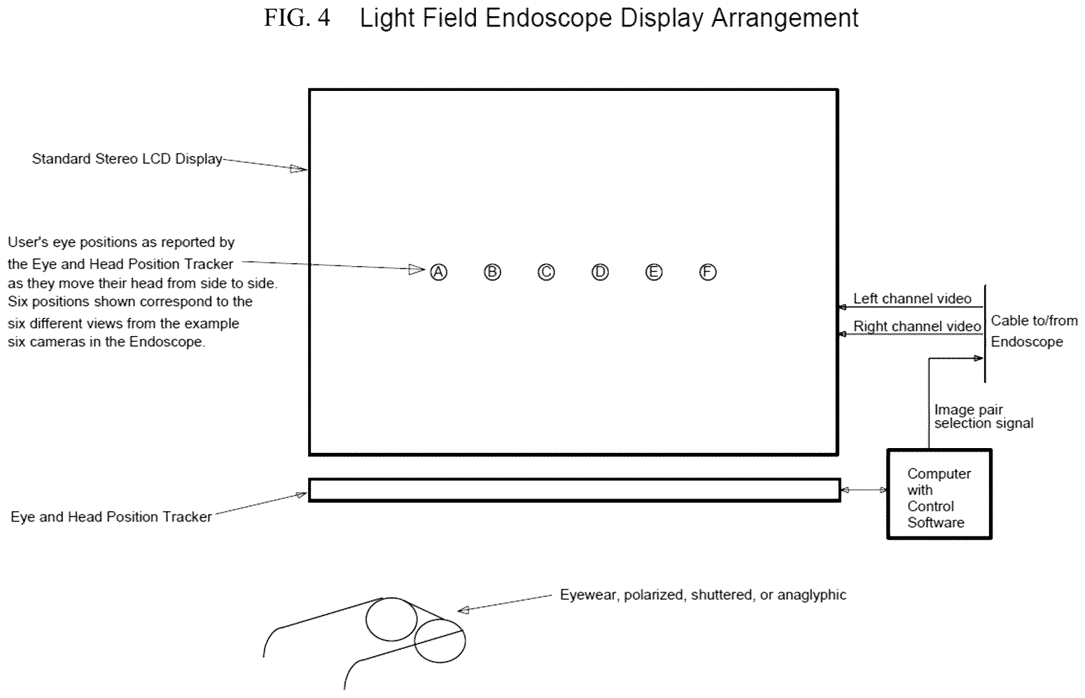

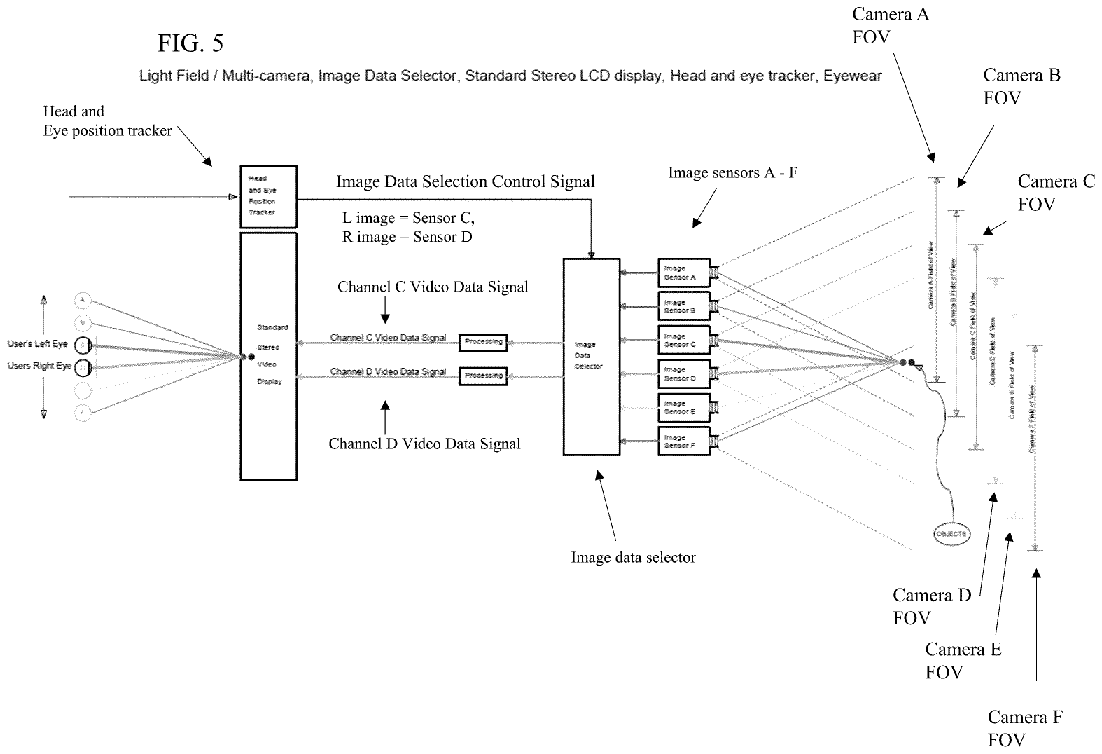

Referring to FIG. 4, the video data streams from the six sensors are sent to a data selector switching system. This selector choses two adjacent images and transmits only those back to the stereo display. An eye and head tracker mounted on the display or positioned beneath or adjacent to the display continuously monitors the user's eye and head position in real time. Assume that the cameras are notated A, B, C, D, E, and F. The pair C and D contain the center stereo view with C corresponding to the left eye view and D the right eye view. This is depicted in FIG. 5 and FIGS. 8A/8B, where the user's left and right eye positions are aligned with C and D respectively. Based on the user's eye and/or head position, the data selector switching system choses images for sensors C and D to display as shown.

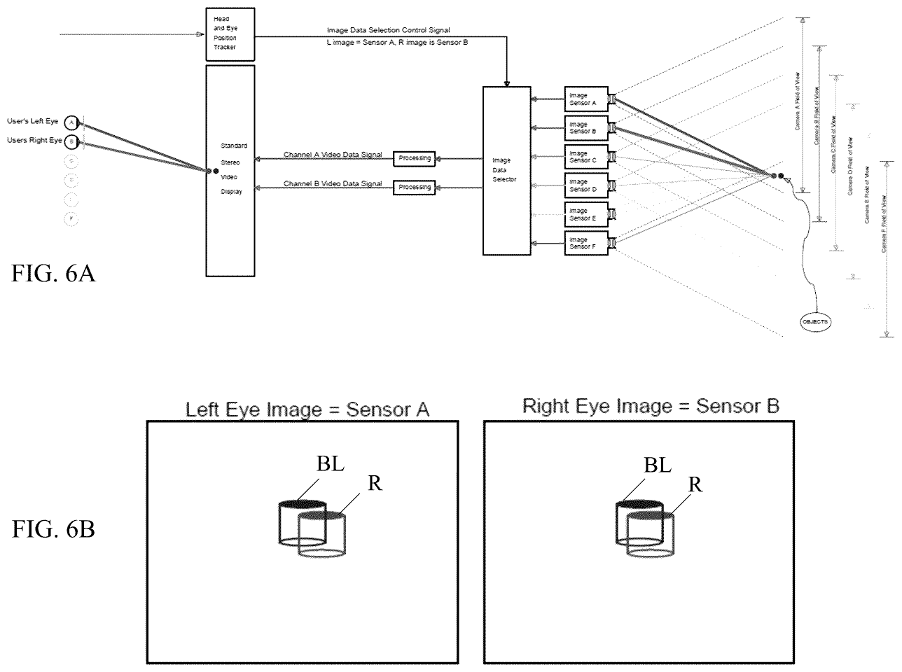

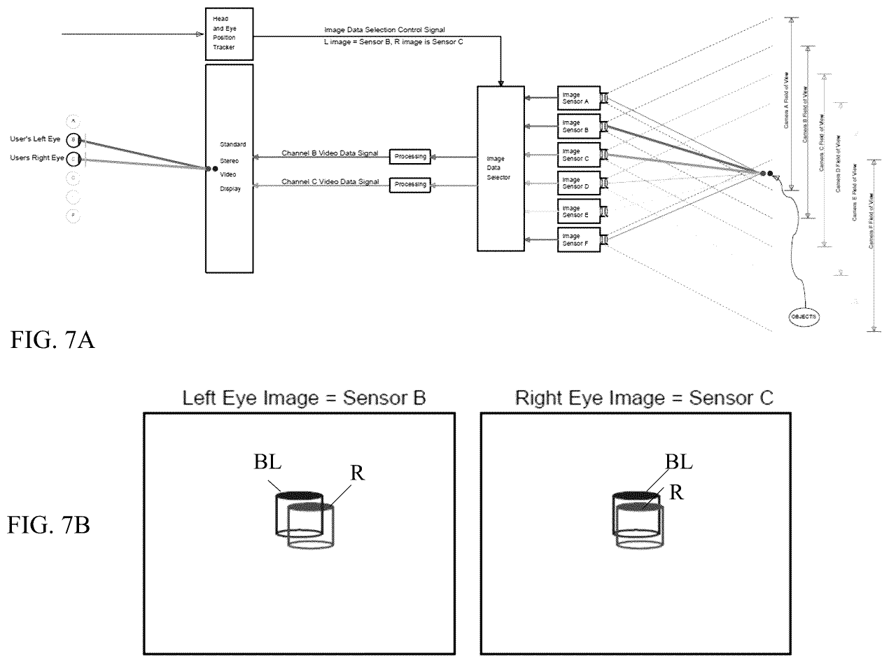

If the user moves their head about one eye to eye spacing to the left, the eye tracker transmits this change to the control software which then sends a signal out to the video switching selector system. This then selects image pair B and C instead. See FIGS. 7A and 7B. Now B is the left eye view and C is the right eye view. If the user moves their head another eye to eye spacing to the left, image pair A and B are selected instead, as shown in FIGS. 6A and 6B. Now A is the left eye view and B is the right eye view.

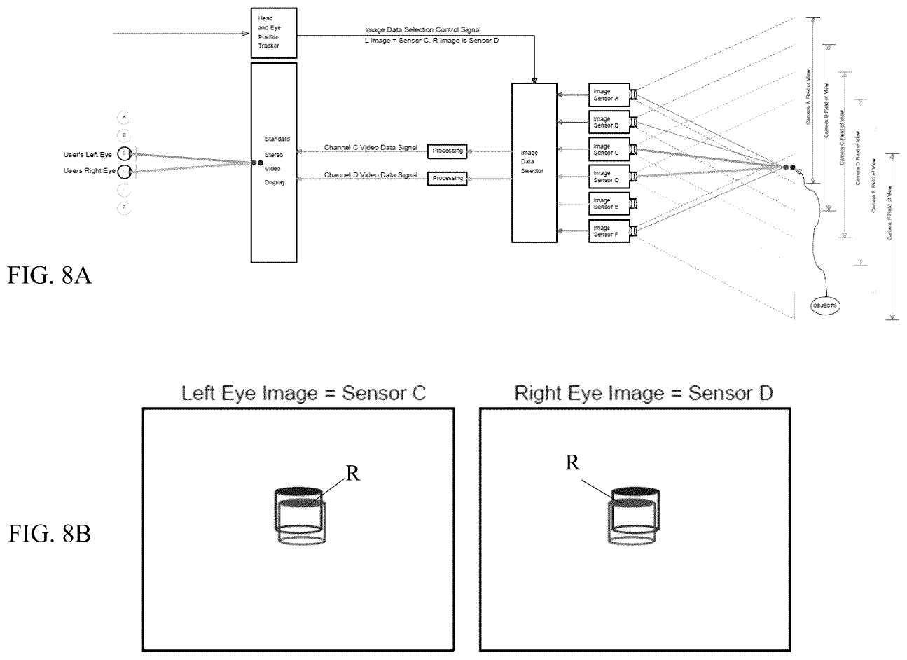

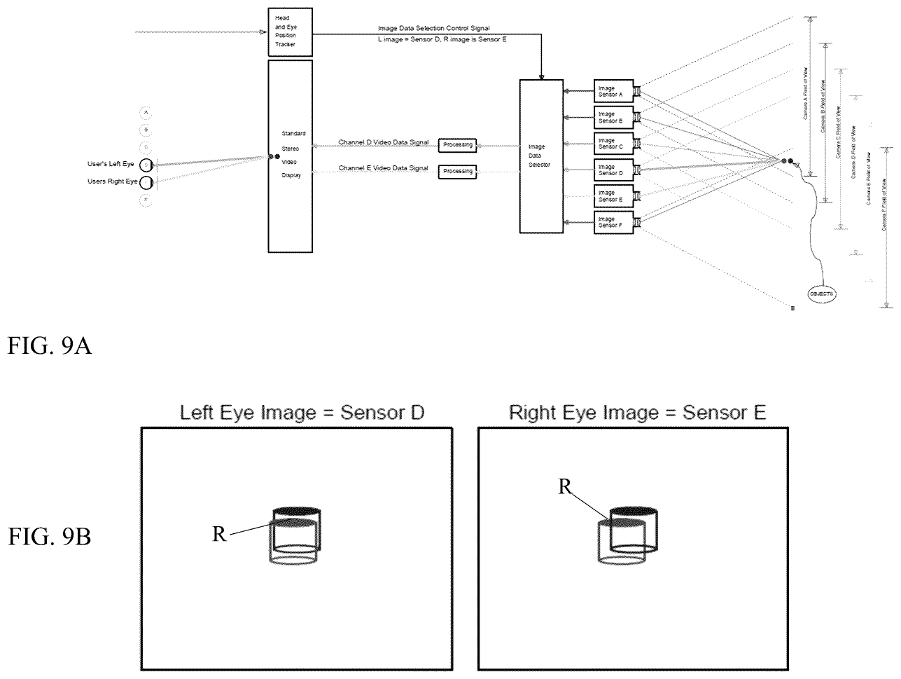

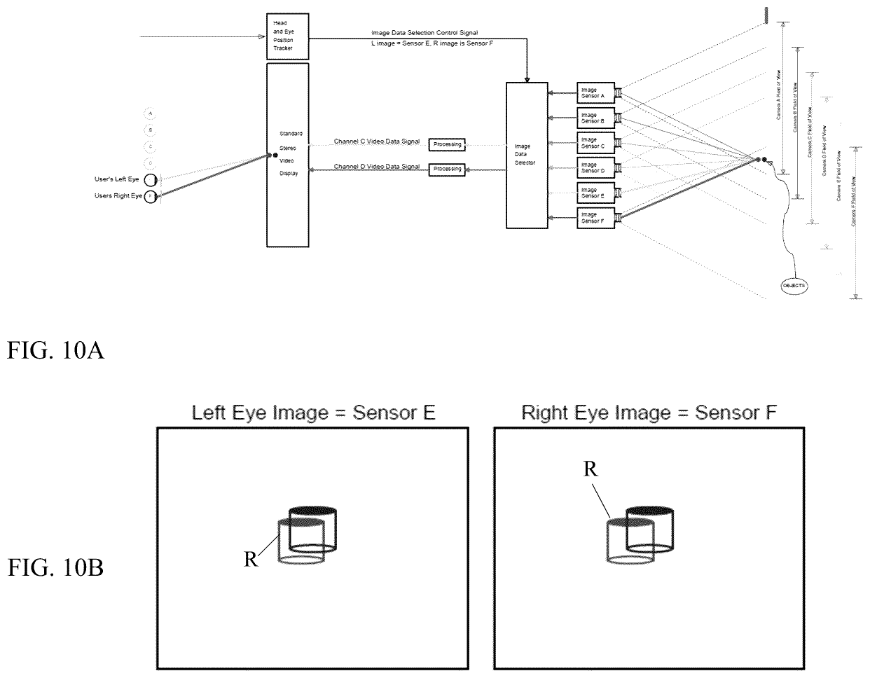

The system works similarly if the user moves their head to the right from center. Views D and E are selected as shown in FIGS. 9A and 9B Now D is the left eye view and E is the right eye view. Finally, if they move their head all the way to the right, E and F are selected as depicted in FIGS. 10A and 10B. Now E is the left eye view and F is the right eye view. Thus, input from the head and eye position tracker is used to detect movement so that as the user moves their head back and forth a different set of views are selected and presented giving the effect of a multi-view autostereoscopic display system but using a simple stereo display.

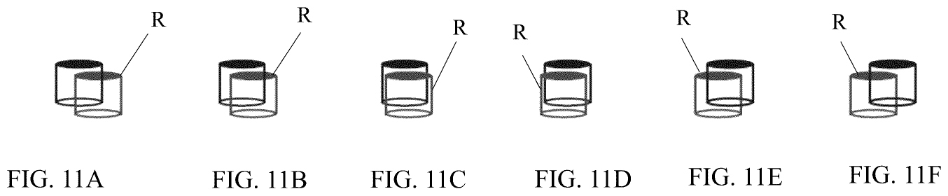

As can be appreciated from a review of FIGS. 6A-11B, which show how different image pairs are selected according to the user eye and head position, in the simplest case, with six image sensors, there are five stereo pairs that can be presented. In each example shown in FIGS. 6B, 7B, 8B, 9B and 10B, the observed object is shown as a red cylinder R in front of a blue cylinder BL. As the eye positions change, the perspective of one in front of the other changes. The image views on the right of the page give insight into the look around capability. This can also be seen in the sequence shown in FIGS. 11A-11F.

In a modification of the disclosed embodiment, the image data from the camera sensors is subjected to image processing that allow the in-between images of stereo pairs to be interpolated. In this embodiment, more than five stereo pairs could be presented based on the six camera sensors, allowing for a smooth transition effect as the user moves their head. In a further modification, a larger number of cameras is used with closer spacing. For example, placing the sensors 2.25 mm apart could result in each stereo pair being two cameras apart, again giving a smoother transition effect during user movements.

The tracking device is preferably both an eye and a head tracker. These may be integrated into a single unit as depicted in FIG. 4, or they may be separate. The trackers may be positioned in proximity to the display as shown. Data from the eye tracker provides data not available from the head tracker, including the distance between the user's eyes, which is used by the system to determine the correct places to switch the image data selector. In alternative embodiments, an eye tracker is used in lieu of the combined head tracker and eye tracker.

Since the purpose of the image data selector is to reduce the number of cables, it would be optimal to place it as close to the camera image sensors as possible. Depending on the sensor data format, this selector could be implemented as a small FPGA or CPLD or analog switch integrated circuit and could be located either on the same board as the image sensors, or it could be located in the shaft of the endoscope or finally it could be located in the box shown at the proximal end of the endoscope. The image sensors should be synchronized with each other so that they all start capturing frames at the same time. The selection control information requires only a small amount of data and thus could easily share the same signal wire as the vertical sync signal. The intention is that the switching from one set of images to another would take place during the vertical scanning interval.

Note that the light source needed to provide illumination for endoscope usage is not shown in any of the figures. This was done for clarity. For this invention, it is assumed that some source of light is included in the light field endoscope system.

A number of advantages are provided by the concepts disclosed in this application. The light field endoscope provides a look around capability that is not provided by current two camera stereo endoscope setups by giving the user the perception of look around 3D capability with horizontal movements of the user's head position. This can allow the user to look around a body cavity without re-positioning the endoscope, adding efficiency to the surgical procedure. For typical robotic surgical applications, robotic manipulators hold and manipulate surgical instruments and endoscopes. Providing an endoscope with look around capability can reduce capital equipment costs by potentially eliminating the need to position the endoscope on a robotic manipulator. Instead, it could be positioned on a fixed support. it can allow the endoscope to be positioned on a stationary device.

The disclosed endoscope does not require a specialized display, but can be used with a standard commercially available stereo display system, eye wear, and an eye tracker device. Moreover, the endoscope does not require significant bandwidth or signal wires as was the case with prior art autostereoscopic systems, but instead offers bandwidth and cable requirements on par with standard endoscope systems.

I claim:

1. An endoscope system comprising:
an endoscopic camera having an elongate shaft, an articulating distal portion, and at least three image sensors, the image sensors positioned to receive light from the articulating distal portion and to generate image data;
at least one of an eye tracker and a head tracker;
an image display;
at least one processor having at least one memory storing instructions executable by the at least one processor to:
receive image data from each of the plurality of image sensors;
receive user view data corresponding to an eye or head position of a user based on signals from the eye tracker or head tracker;
in response to the user view data, selecting image data from only two of the at least three image sensors and causing the selected image data to be displayed on the image display.

2. The system of claim 1, wherein the endoscopic camera includes between 3 and 6 image sensors.

3. The system of claim 1, wherein the articulating portion is moveable between a first position in which a longitudinal axis of the shaft is longitudinally aligned with a longitudinal axis of the articulating portion, and a second position in which the longitudinal axis of the articulating portion extends angularly to the longitudinal axis of the shaft.

4. The system of claim 3, wherein in the second position the longitudinal axis of the articulating portion is orthogonal to the longitudinal axis of the shaft.
